(19) United States
(12) Patent Application Publication
(10) Pub. No.: US 2024/0148761 A1
(43) Pub. Date: May 9, 2024

(54) Chelated Metal Oxide Cannabinoid Gel Composition

(71) Applicant: William Wingfield, Henrico, VA (US)

(72) Inventor: William Wingfield, Henrico, VA (US)

(21) Appl. No.: 18/387,007

(22) Filed: Nov. 3, 2023

Related U.S. Application Data
(60) Provisional application No. 63/422,114, filed on Nov. 3, 2022.

(51) Int. Cl. A61K 31/00; A61K 31/722; A61K 33/38

(52) U.S. Cl. CPC A61K 31/658; A61K 31/722; A61K 33/38; A61K 2121/00

(57) ABSTRACT

A homogeneous gel composition for topical wound treatment and other healthcare purposes. Said gel composition comprising a cationic chelated chitosan-silver gel incorporating at least one cannabinoid and having antimicrobial, antibacterial, anti-inflammatory and/or analgesic properties. A method for treating a wound or skin disorder using the gel composition is also disclosed.

### CROSS-REFERENCE TO RELATED APPLICATIONS

This application claims the benefit of the filing date of U.S. Provisional Patent Application Ser. No. 63/422,114 filed on Nov. 3, 2022.

### FIELD OF THE INVENTION

The invention relates to topical anti-microbial, anti-inflammatory, and/or analgesic compositions for wound and skin care and treatment.

### BACKGROUND OF THE INVENTION

Wound healing is a complex biological process that involves the restoration of the skin matrix due in response to disease or injury. This process can be complicated by infection of the wound/lesion site which can significantly inhibit healing time and also can affect the quality and strength of tissue that is regenerated. More effective methods and compositions are always needed to improve would healing and related physiological processes.

The four stages of wound healing include hemostasis, inflammatory, proliferation and remodeling. In hemostasis, the trauma of a wound causes the body to initiate blood vessel restriction and directs platelets to the site. Blood clotting forms a provisional temporary matrix at the wound site. Simultaneously, stimulation, migration and adhesion of fibroblasts, keratinocytes and endothelial cells occur. The inflammatory phase results in the production of histamine and is accompanied by swelling and pain. In the inflammatory phase, macrophages increase the rate of dermal regeneration and skin repair at the wound location. The role of macrophages in wound healing may be related to their production of nitric oxide. Next, in the proliferation phase, the rebuilding of damaged tissue occurs through the development of a temporary collagen matrix composed primarily of fibroblasts. This temporary matrix is known as granulation tissue. During the final remodeling phase, the granulation tissue matures to formed tissue matrix.

Among the various substances used to accelerate and positively enhance the wound healing process and reduce complications, silver and silver salts have been used as antimicrobial agents for many years. Silver salts, colloids, and complexes have been used to prevent and to control infection. More specifically, in one application, silver oxide may be successfully incorporated into gel compositions to provide anti-microbial benefits for enhanced wound hearing. When manufacturing such compositions containing silver, the use of metal oxide chelation can be beneficial as it permits incorporation of higher and more stable concentrations of silver-oxide particles in the final product.

Chitosan, a deacetylated homopolymer of acetyl-glucosamine, is sometimes used topically to treat wounds. Indeed, it is well known that chitosan itself possesses multiple properties which are beneficial to the healing process. Moreover, chitosan and its derivatives can be used as chelating agents in the production of silver-oxide gel compositions as discussed above.

Chitosan developed from fungal chitin has substantially different molecular characteristics from chitosan developed from crustacean chitin that results in the fungal chitosan having an enhanced bioactivity. For example, the particle size of the fungal chitosan is smaller enabling the fungal chitosan to chelate silver ions more effectively. The fungal chitosan contains more receptor sites to which silver can bind and binds the silver more tightly. Consequently, the compositions of the present invention comprising a chelated fungal chitosan-silver complex as described herein can have enhanced bioactivity such as increased antimicrobial activity and increased wound healing activity.

Finally, there is a tremendous interest in the use of Cannabis sativa and its active ingredients for the treatment of a variety of medical conditions. With regard to wound care, studies have shown that certain phytocannabinoids, in particular, delta-9-tetrahydrocannabinol (THC) and cannabidiol (CBD) can have a highly beneficial effect on wound healing. Studies have clearly demonstrated that the topical application of such cannabinoids can have anti-inflammatory, anti-itching, analgesic, wound healing and anti-proliferative effects on human skin.

It would therefore be beneficial to have a single wound care composition which incorporates all of the foregoing elements and benefits in therapeutically effective amounts.

### SUMMARY OF THE INVENTION

In the primary embodiment, a homogeneous gel composition with at least one anti-inflammatory and or anti-pain cannabinoid, namely, cannabidiol (commonly known as CBD), tetrahydrocannabinol (commonly known as THC), or a combination of both, and having antimicrobial, antibacterial, and/or anti-viral properties is disclosed. Such a gel composition comprises a combination of a stable and homogeneous aqueous and/or a polyol solution based chelated metal oxide liquid complex suspension and a gel composition, wherein the homogeneous gel composition contains chelated metal oxide particles suspended therein.

Further, a method of enhancing the healing of a wound in a subject is provided. The method comprising contacting a wound in the subject with a therapeutically effective amount of a cationic solution, gel composition, or other pharmaceutical composition of the invention disclosed herein and thereby enhancing healing of the wound in the subject or otherwise providing beneficial or desired effects.

### DESCRIPTION OF THE DRAWINGS

FIG. 1 is process diagram which identifies the steps of manufacture of the invention disclosed herein.

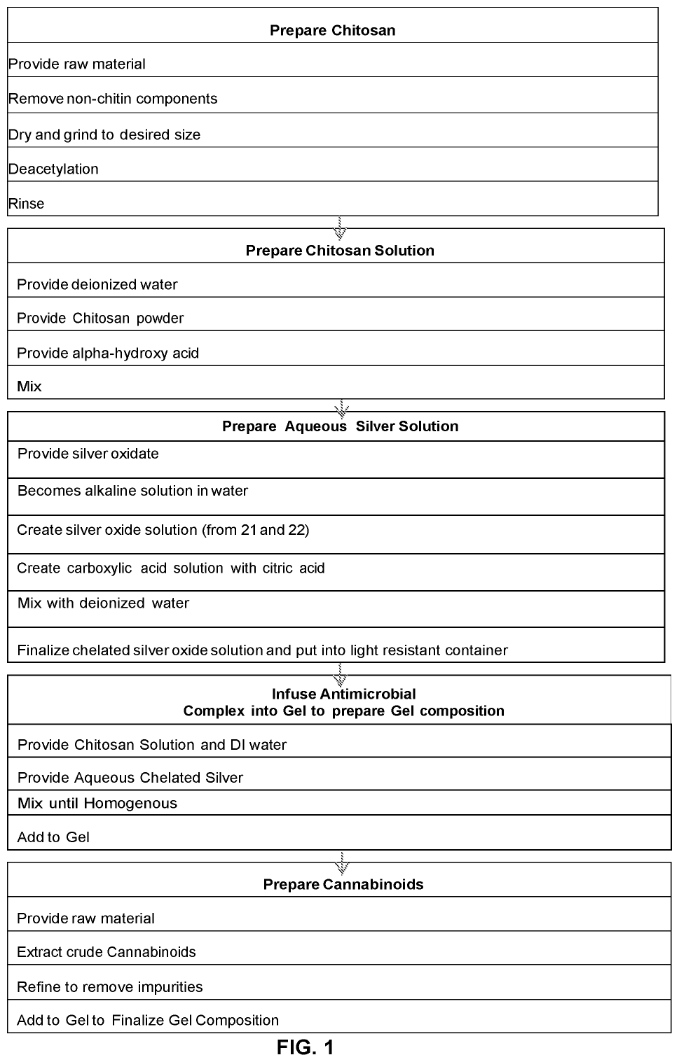

### DETAILED DESCRIPTION

In the preferred embodiment, the present invention is a homogeneous gel composition with at least one anti-inflammatory and/or anti-pain cannabinoid, e.g., cannabidiol (commonly known as CBD) or tetrahydrocannabinol (commonly known as THC) or a combination of both, having antimicrobial, antibacterial, and/or anti-viral properties. The homogenous gel comprises a combination of a stable and homogeneous aqueous and/or polyol solution based chelated metal oxide liquid complex suspension and a gel composition free of chelated metal oxides comprising water, polyquaternium, a dihydroxy alcohol, and hyaluronic acid, wherein the homogeneous gel composition contains chelated metal oxide particles (notably silver oxide, copper oxide, zinc oxide, gold oxide) homogeneously suspended in the homogeneous gel composition. The concentration of the chelated metal oxide particles contained within the homogeneous gel being in a concentration of at least 0.001 weight percent (10 ppm). In a preferred embodiment, the cannabinoids are homogeneously suspended in the homogeneous gel composition such that a concentration of the cannabinoids contained within the homogeneous gel are in a concentration of at least 0.25 percent.

Chitosan developed from fungal chitin is preferable. The fungal chitosan may be 0.1% to 30% by weight of the solution. The chelated fungal chitosan-silver complex may comprise fungal chitosan chelated with 1 ppm to 1,000 ppm silver. In a first embodiment, tetrahydrocannabinol may be 0.001% to 45% by weight of the gel. In an alternate embodiment, cannabidiol may be 0.001% to 45% by weight of the gel.

In a preferred embodiment, the percentages of elements in a gel composition would be essentially as follows: Water (preferably distilled) 76%; Polyquaterniums (Cationic Biopolymers) 5.3%; Chelated Metal Oxides suspension (Chitosan-Ag) 5%; Glycerin 9%; Arginine 2% (if pH buffering is required); Cannabinoids (CBD either broad spectrum, full spectrum or crystalline isolate or THC oil or crystalline isolate) 1%; and Hyaluronic Acid 1.7%.

The invention further provides a composition comprising the cationic gel of the invention and a pharmaceutically acceptable carrier. “Pharmaceutically acceptable,” as used herein, refers to a material that can be administered to an individual along with the gel composition disclosed herein without causing substantial deleterious biological effects or negatively interacting with any other components of the gel composition. In some embodiments, a composition of the invention may include a pharmaceutically acceptable carrier. Examples of such carriers include, but are not limited to, petroleum jelly, polysaccharides, lanolines, glycols, glycerides, alcohols, transdermal enhancers, and combinations thereof.

Cannabinoids, tetrahydrocannabinol (THC) and/or cannabidiol (CBD) can be extracted by, but not limited to, methods of extraction incorporating ice water, alcohol, CO2, heat & pressure (rosin press), kief (grinding & pressing), oil or water vapor. The resulting Cannabinoid extract whether THC or CBD full spectrum, broad spectrum, isolate, etc. can be incorporated into the invention. By way of example, an extracted cannabinoid oil may be added to the polyquaternium/polymeric gel.

The preferred manner of formulating the homogeneous gel composition involves the combination of three groups of ingredients. Group A consists of an aqueous solution of polyquaterniums (cationic polyelectrolytes), water, glycerin and hyaluronic acid. Group B consists of chitosan-Ag in a concentration of 10 to 1,000 ppm. Group C consists of cannabidiol (CBD) or tetrahydrocannabinol or tetrahydrocannabinol (THC) in a concentration of at least 0.25 percent. Each respective group is separately composed initially. Group B is then introduced into Group A under agitation and then the combination is introduced into followed Group C under agitation. The order of addition may be altered by introducing Group C into Group A followed by Group B under agitation. Preferred means of agitation is by moderate speed planetary or sweep action mixer, but other mixing methods may be employed, such as high speed emulsion, sonic, turbulent, vortex, double cone, plough, ribbon, high and low viscosity, shear, blender, disperser, propeller, conical screw, v-type blender, baffle, impeller, air-jet, fluid jet, radial, axial, tangential flow, hand-held, stirrer, agitator, slant, tumbler, portable (mixer) etc.

The cationic gels, and compositions thereof, may be used to promote healing of soft tissue or ulcers, encourage the migration of epithelial and fibroblasts to wound sites, accelerate the recovery of damaged neural tissue, reduce edema, and inhibit the growth of pathogens through application of a therapeutically effective dose. The therapeutically effective dose being such amount that is adequate to provide improvement or benefit to the subject with regard to the area being treated or the alleviation of at least one clinical symptom arising from the wound or skin disorder being treated with the invention.

While the invention has been described in reference to certain preferred embodiments, it will be readily apparent to one of ordinary skill in the art that certain modifications or variations may be made to the system without departing from the scope of invention claimed below and described in the foregoing specification.

What is claimed is:

1. A cationic gel for wound care comprising a cannabinoid chitosan-silver complex composition.

2. A cationic gel composition for use on skin comprising:
fungal chitosan chelated with about 1 ppm to about 1,000 ppm silver, and
at least one cannabinoid.

3. The cationic gel composition of claim 2 wherein said at least one cannabinoid is tetrahydrocannabinol or cannabidiol.

4. A method for treating a wound or skin disorder, the method comprising topically administering to a tissue of a subject in need thereof a therapeutically effective quantity of a cannabinoid chelated fungal chitosan-silver complex composition.
